(19) United States
(12) Patent Application Publication
(10) Pub. No.: US 2025/0176980 A1
(43) Pub. Date: Jun. 5, 2025

(54) Apparatus for Guiding Fixation of Distal Tibiofibular Joint

(71) Applicant: Sanford Health, Sioux Falls, SD (US)

(72) Inventor: Tim Uglem, Sioux Falls, SD (US)

(21) Appl. No.: 18/969,160

(22) Filed: Dec. 4, 2024

Related U.S. Application Data
(60) Provisional application No. 63/606,426, filed on Dec. 5, 2023.

(51) Int. Cl. A61B 17/17

(52) U.S. Cl. CPC A61B 17/1775

(57) ABSTRACT

An apparatus includes a base and a first arm extending from the base. The first arm includes a first clamping end configured to conform to a fibula. The apparatus also includes a second arm extending from the base opposite the first arm, the second arm including a second clamping end configured to conform to the fibula. The apparatus also includes a compression mechanism configured to press the first clamping end toward the second clamping end to grasp the fibula between the first clamping end and the second clamping end. The apparatus also includes a guide tube between the first arm and the second arm. The guide tube is formed of a radiodense material and is configured to receive and direct a guide pin for insertion through the fibula and into a tibia.

### CROSS REFERENCE TO RELATED APPLICATION

This application claims priority to U.S. provisional application No. 63/606,426, filed on Dec. 5, 2023, the entire contents of which are hereby incorporated by reference herein.

### BACKGROUND

Ankle fractures are very common. Repairing some ankle fractures can involve repairing the distal tibiofibular syndesmosis by fixing the fibula to the tibia with a screw or another fixation device. Properly aligning the distal tibiofibular joint while applying the fixation device between the fibula and the tibia can be difficult.

### SUMMARY

A first example includes an apparatus comprising: a base; a first arm extending from the base and comprising a first clamping end configured to conform to a fibula; a second arm extending from the base opposite the first arm, the second arm comprising a second clamping end configured to conform to the fibula; a compression mechanism configured to press the first clamping end toward the second clamping end to grasp the fibula between the first clamping end and the second clamping end; and a guide tube between the first arm and the second arm, wherein the guide tube is formed of a radiodense material and is configured to receive and direct a guide pin for insertion through the fibula and into a tibia.

A second example includes a method of using the apparatus of the first example, the method comprising: placing the apparatus on the fibula in an aligned position such that the guide tube is substantially aligned with an anatomic axis of a distal tibiofibular joint that includes the fibula and the tibia; engaging the compression mechanism while the apparatus is in the aligned position to press the first clamping end toward the second clamping end to grasp the fibula; and inserting the guide pin through the guide tube, through the fibula, and into the tibia while the apparatus is in the aligned position.

When the term “substantially” or “about” is used herein, it is meant that the recited characteristic, parameter, or value need not be achieved exactly, but that deviations or variations, including, for example, tolerances, measurement error, measurement accuracy limitations, and other factors known to those of skill in the art may occur in amounts that do not preclude the effect the characteristic was intended to provide. In some examples disclosed herein, “substantially” or “about” means within +/−0-5% of the recited value.

### BRIEF DESCRIPTION OF THE FIGURES

The above, as well as additional, features will be better understood through the following illustrative and non-limiting detailed description of example embodiments, with reference to the appended drawings.

FIG. 1 is a block diagram of an apparatus, according to an example.

FIG. 2 is a schematic distal view of an apparatus, a fibula, a tibia, and a talus, according to an example.

FIG. 3 is a schematic cross section of an apparatus, a fibula, and a tibia, according to an example.

FIG. 4 is a schematic cross section of a guide pin, a fibula, and a tibia, according to an example.

FIG. 5 is a schematic cross section of a fibula and a tibia, according to an example.

FIG. 6 is a schematic cross section of a fixation device, a fibula, and a tibia, according to an example.

FIG. 7 is a schematic distal view of a surgical plate, a guide pin, a fibula, a tibia, and a talus, according to an example.

FIG. 8 is an x-ray image depicting proper alignment of a guide pin, a fibula, and a tibia, according to an example.

FIG. 9 is an x-ray image depicting improper alignment of a guide pin, a fibula, and a tibia, according to an example.

FIG. 10 is an x-ray image depicting improper alignment of a guide pin, a fibula, and a tibia, according to an example.

FIG. 11 is an x-ray image depicting improper alignment of a guide pin, a fibula, and a tibia, according to an example.

FIG. 12 is an x-ray image depicting improper alignment of a guide pin, a fibula, and a tibia, according to an example.

FIG. 13 is a block diagram of a method, according to an example.

All the figures are schematic, not necessarily to scale, and generally only show parts which are necessary to elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example. Furthermore, like numbers refer to the same or similar elements or components throughout.

As noted above, a need exists for an apparatus and a method for more reliably aligning the distal tibiofibular joint while applying a fixation device between the fibula and the tibia. This disclosure includes an apparatus that includes a base, a first arm, and a second arm. The first arm extends from the base and includes a first clamping end configured to conform to the fibula. The second arm extends from the base opposite the first arm and includes a second clamping end configured to conform to the fibula. The apparatus also includes a compression mechanism configured to press the first clamping end toward the second clamping end to grasp the fibula between the first clamping end and the second clamping end. The apparatus also includes a guide tube between the first arm and the second arm. The guide tube is formed of a radiodense material and is configured to receive and direct a guide pin for insertion through the fibula and into a tibia.

This disclosure also includes a method of using the apparatus. The method includes placing the apparatus on the fibula in an aligned position such that the guide tube is substantially aligned with an anatomic axis of a distal tibiofibular joint that includes the fibula and the tibia. The method also includes engaging the compression mechanism while the apparatus is in the aligned position to press the first clamping end toward the second clamping end to grasp the fibula. The method also includes inserting the guide pin through the guide tube, through the fibula, and into the tibia while the apparatus is in the aligned position.

The orientation of the guide tube, the fibula, and the tibia can be monitored in real time using x-ray fluoroscopy so that the aligned position is verified before fully clamping the apparatus into place on the fibula. This can help place the guide pin through the guide tube, the fibula, and the tibia properly such that a fixation device can be applied to the fibula and the tibia while in the properly aligned position.

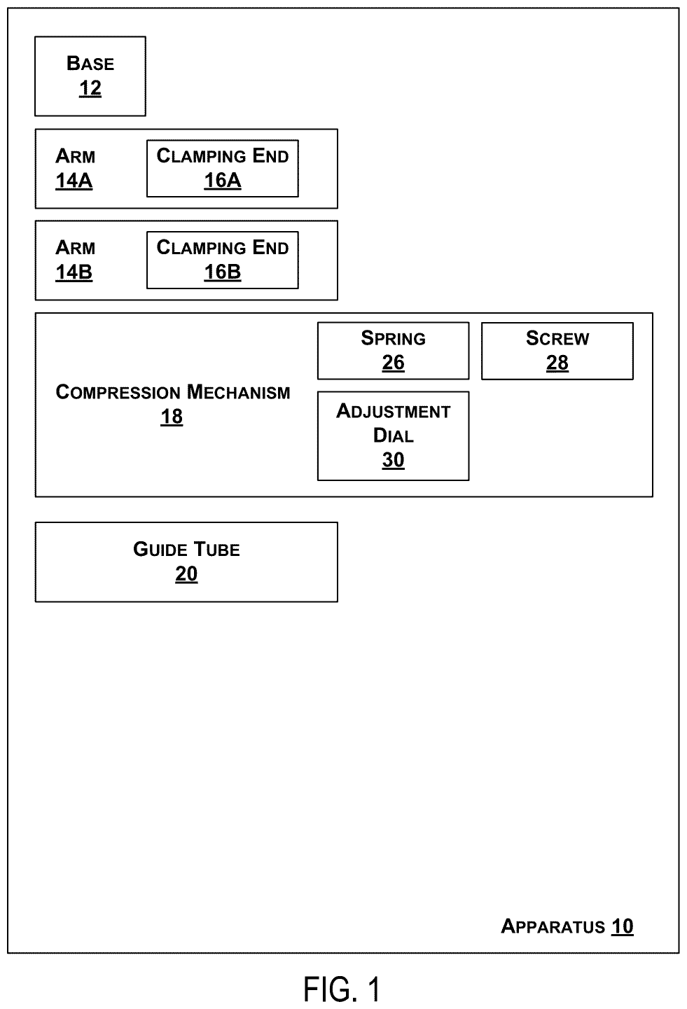

FIG. 1 is a block diagram of an apparatus 10. The apparatus 10 includes a base 12, an arm 14A, an arm 14B, a compression mechanism 18, and a guide tube 20.

The arm 14A includes a clamping end 16A configured to conform to a fibula.

The arm 14B includes a clamping end 16B configured to conform to the fibula.

The compression mechanism 18 includes one or more of a spring 26, a screw 28, or an adjustment dial 30, each of which are configured alone or in concert to press the clamping end 16A toward the clamping end 16B to grasp the fibula between the clamping end 16A and the clamping end 16B.

The guide tube 20 is formed of a radiodense material such as aluminum, stainless steel, or another metal and is configured to receive and direct a guide pin for insertion through the fibula and into a tibia.

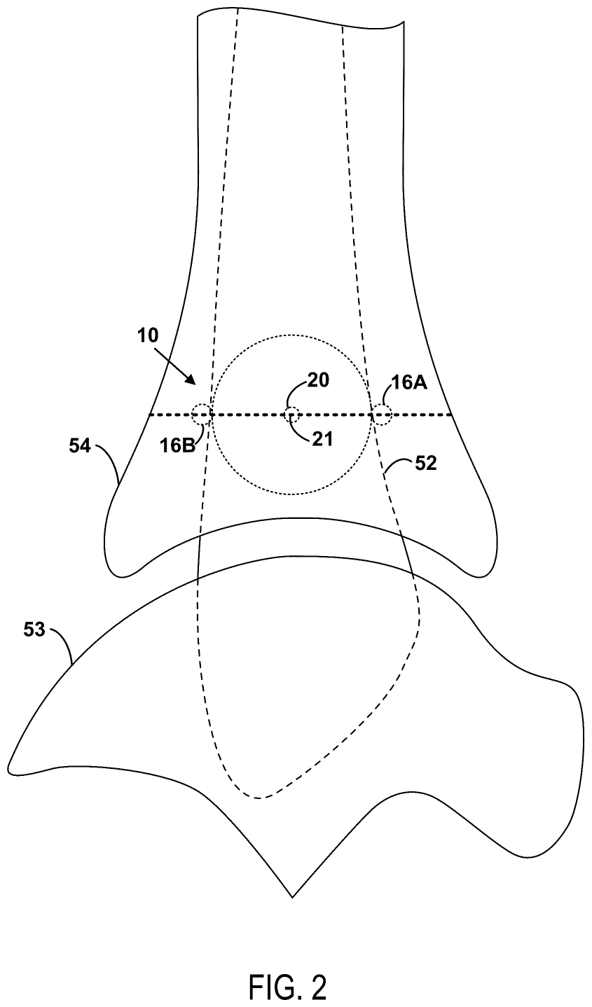

FIG. 2 is a schematic distal view of a tibia 54, a fibula 52, a talus 53, and the apparatus 10. The fibula 52 and the apparatus 10 are shown with hashed lines and are partially or fully obscured by the tibia 54 or the talus 53. FIG. 2 shows the clamping end 16A, the clamping end 16B, the guide tube 20, and an anatomic axis 21.

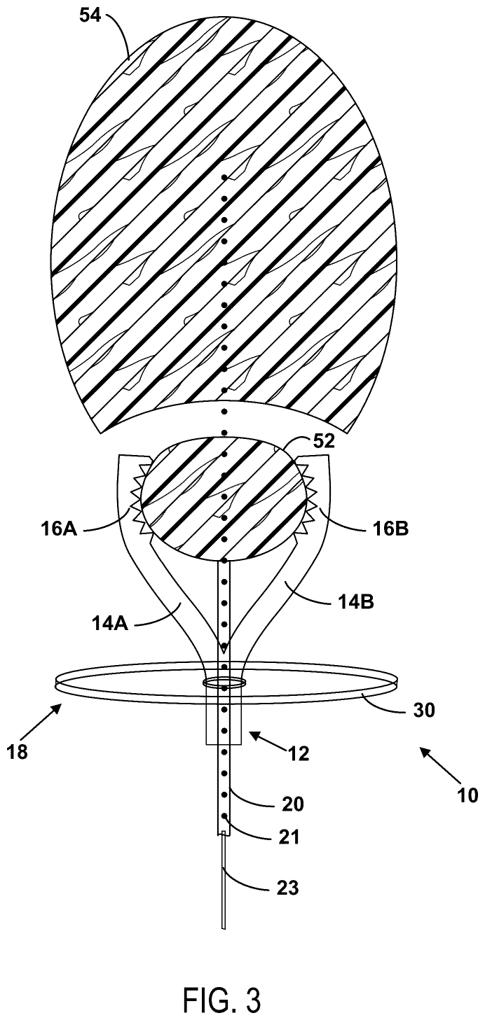

FIG. 3 is a schematic diagram of the apparatus 10, the fibula 52, and the tibia 54. More specifically, FIG. 3 shows a cross section of the fibula 52 and the tibia 54 above the apparatus 10. FIG. 3 also shows the anatomic axis 21 of a distal tibiofibular joint formed by the fibula 52 and the tibia 54.

The apparatus 10 includes the base 12, the arm 14A, the arm 14B, the compression mechanism 18, and the guide tube 20. The arm 14A extends from the base 12 and includes the clamping end 16A configured to conform to and grip the fibula 52. The arm 14B extends from the base 12 opposite the arm 14A and includes the clamping end 16B configured to conform to and grip the fibula 52. As shown, the arm 14A and the arm 14B are joined together at the base 12. The clamping end 16A and the clamping end 16B both have a serrated surface configured to conform to and grip the fibula 52. The base 12, the arm 14A, and the arm 14B are generally formed of radiolucent materials, but other examples are possible.

The compression mechanism 18 takes the form of the adjustment dial 30 in FIG. 3. The compression mechanism 18 is configured to press the clamping end 16A toward the clamping end 16B to grasp the fibula 52 between the clamping end 16A and the clamping end 16B. The adjustment dial 30 has a disc shape and includes a hole through which the base 12 is inserted. For example, the exterior surface of the base 12 and the interior surface of the adjustment dial 30 could be coupled together via a threaded connection. Turning the adjustment dial 30 around the base 12 could increase or decrease a level of compression applied by the arm 14A and the arm 14B to the fibula 52. The compression mechanism 18 can be formed of radiodense and/or radiolucent materials.

Additionally or alternatively, the compression mechanism 18 includes the spring 26 that applies a pre-loaded compression force to the arm 14A and the arm 14B, and thereby to the fibula 52. Additionally or alternatively, the compression mechanism 18 includes the screw 28 that can be manually turned to adjust a level of compression applied by the arm 14A and the arm 14B to the fibula 52.

The guide tube 20 is positioned between the arm 14A and the arm 14B through the base 12. The guide tube 20 is formed of a radiodense material such as stainless steel, aluminum, and/or another metal and is configured to receive and direct a guide pin 23 for insertion through the fibula 52 and into the tibia 54. That is, the guide tube 20 takes the form of a hollow cylinder configured to receive and direct the guide pin 23 for insertion through the fibula 52 and into the tibia 54.

A user such as a surgeon can place the clamping end 16A and the clamping end 16B of the apparatus 10 on opposite sides of the fibula 52 in an aligned position such that the guide tube 20 is substantially aligned with the anatomic axis 21. The user can rotate or move the apparatus 10, for example within a plane that is parallel to the cross sectional plane of FIG. 3, until the aligned position is achieved, that is, until the guide tube 20 is substantially aligned with the anatomic axis 21. The guide tube 20 is generally fixed within the base 12, so movement of the base 12, the arm 14A, and the arm 14B can determine the orientation and position of the guide tube 20. Typically, the surgeon uses real time x-ray fluoroscopy to determine when the apparatus 10 is in the aligned position. In some examples, the user repairs a fracture of the fibula 52 and/or reduces the syndesmosis prior to placing the apparatus 10 on the fibula 52.

While the apparatus 10 is in the aligned position, the user engages the compression mechanism 18 to press the clamping end 16A toward the clamping end 16B to grasp the fibula 52. As noted above, engaging the compression mechanism 18 can involve turning the adjustment dial 30 around the base 12 and/or turning the screw 28, with or without the aid of the spring 26. While the apparatus 10 is still in the aligned position, the user inserts the guide pin 23 through the guide tube 20, through the fibula 52, and into the tibia 54. The guide pin 23 is generally inserted such that the guide pin 23 is substantially aligned with the anatomic axis 21.

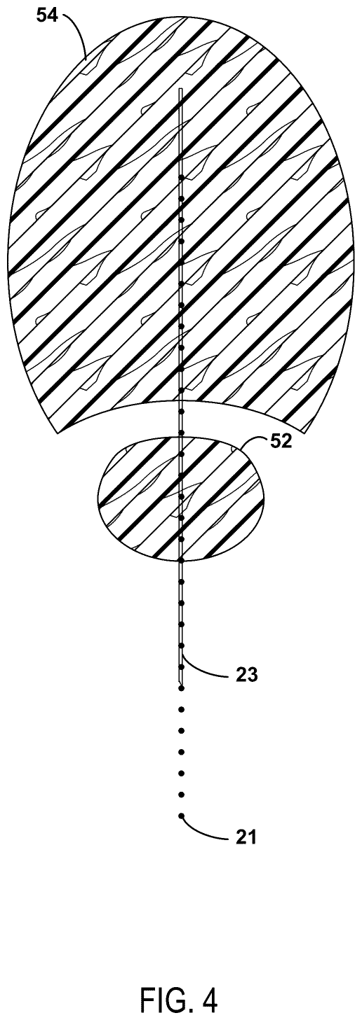

In some examples, the guide pin 23 is inserted by drilling the guide pin 23 through the guide tube 20, through the fibula 52, and into the tibia 54 such that the guide pin 23 extends distally from the fibula 52 as shown in FIG. 4.

FIG. 4 is a schematic diagram of the fibula 52, the tibia 54, and the guide pin 23 after the apparatus 10 has been removed. As shown, the guide pin 23 extends distally from the fibula 52 along the anatomic axis 21. The guide pin 23 is also embedded in the tibia 54 along the anatomic axis 21.

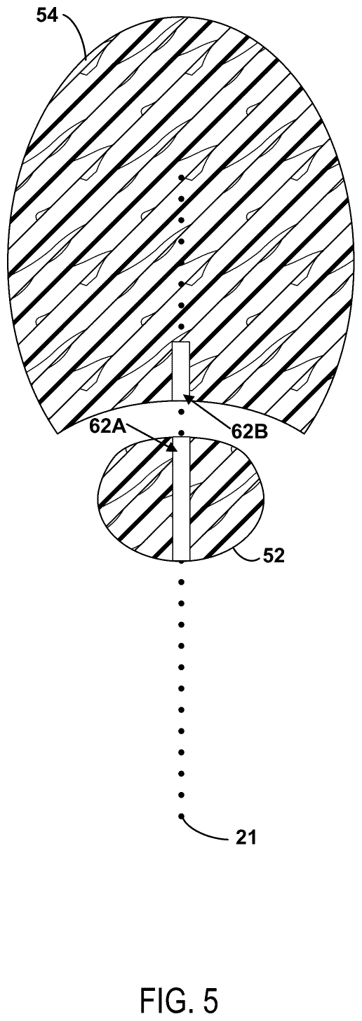

As shown in FIG. 5, the user can drill out the guide pin 23 through the fibula 52 and into the tibia 54 to create a cavity 62A in the fibula 52 and a cavity 62B in the tibia.

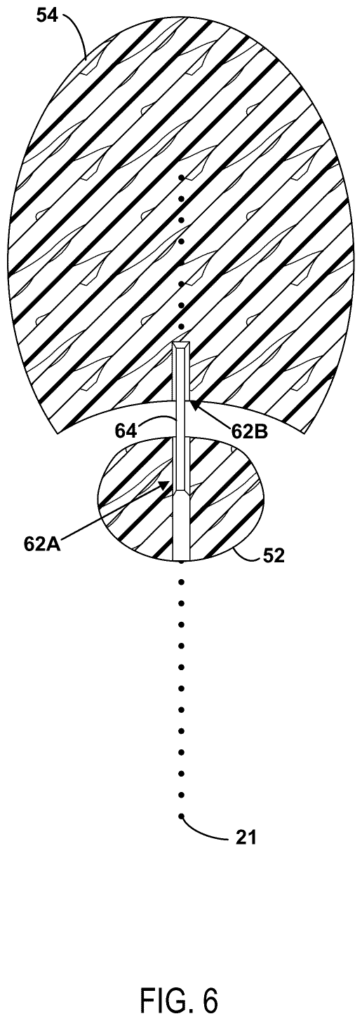

As shown in FIG. 6, the user can bind the fibula 52 to the tibia 54 using a fixation device 64 that is inserted into the cavity 62A and the cavity 62B. In some examples, the fixation device 64 comprises a screw and/or a suture.

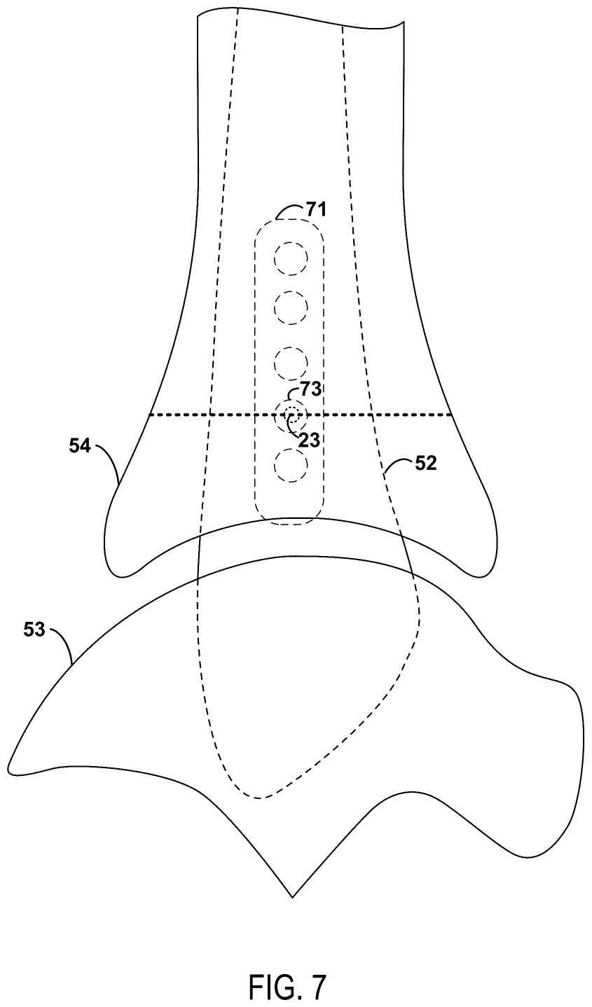

In an example shown in FIG. 7, prior to drilling out the guide pin 23, the user can place a surgical plate 71 on a distal side of the fibula 52 such that the guide pin 23 extends distally through a hole 73 in the surgical plate 71. In some examples, the guide pin 23 also passes through a guide sleeve that is inserted into the hole 73.

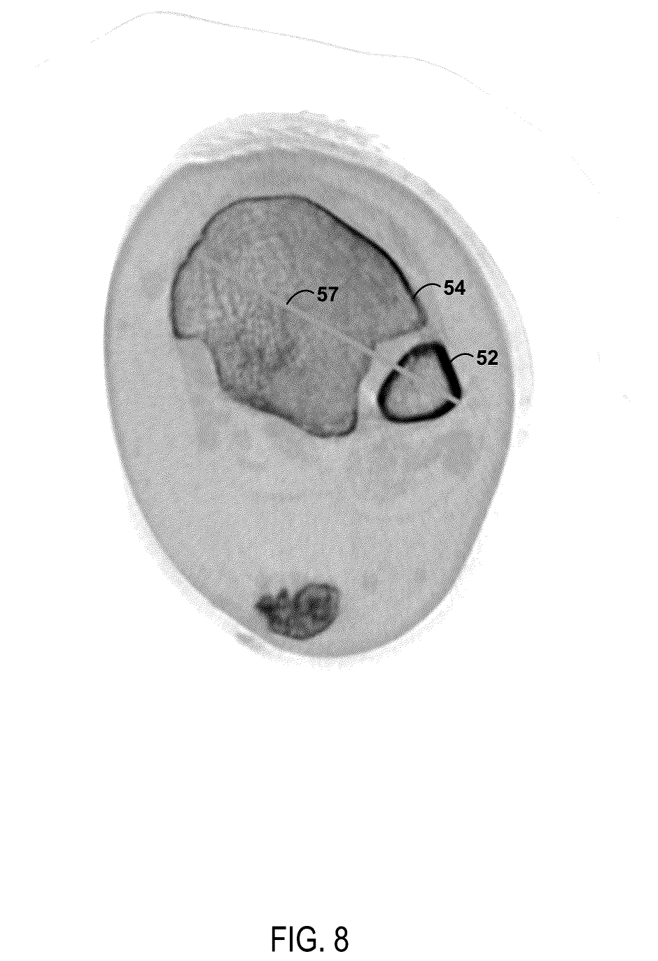

FIG. 8 shows the fibula 52 and the tibia 54. The line 57 coincides with the anatomical axis 21. Thus, if the guide tube 20 is aligned with the line 57, the guide pin 23 inserted through the guide tube 20 will be properly aligned with the fibula 52 and the tibia 54.

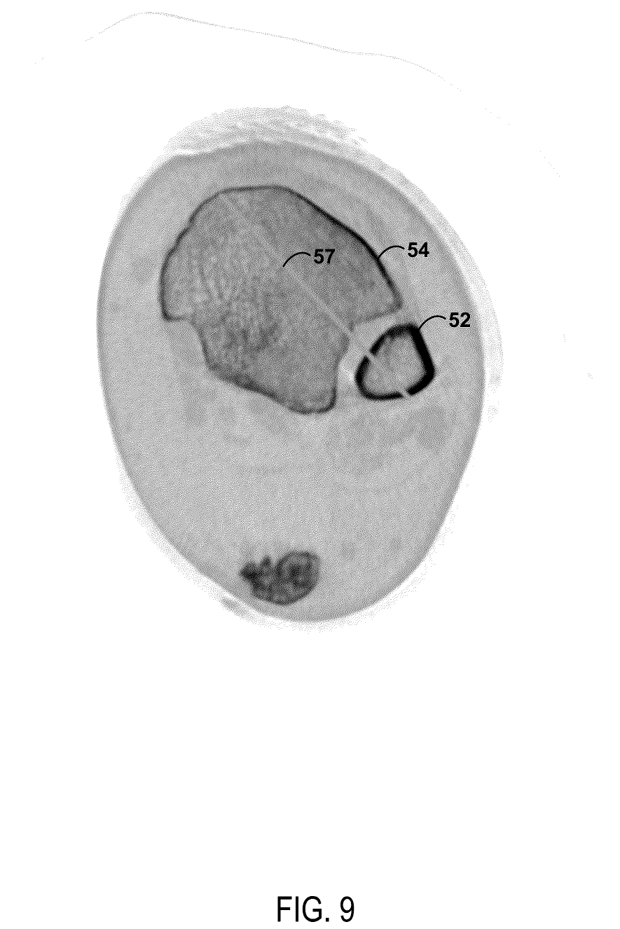

FIG. 9 shows the fibula 52 and the tibia 54. The line 57 does not coincide with the anatomical axis 21. Thus, if the guide tube 20 is aligned with the line 57, the guide pin 23 inserted through the guide tube 20 will not be properly aligned with the fibula 52 and the tibia 54.

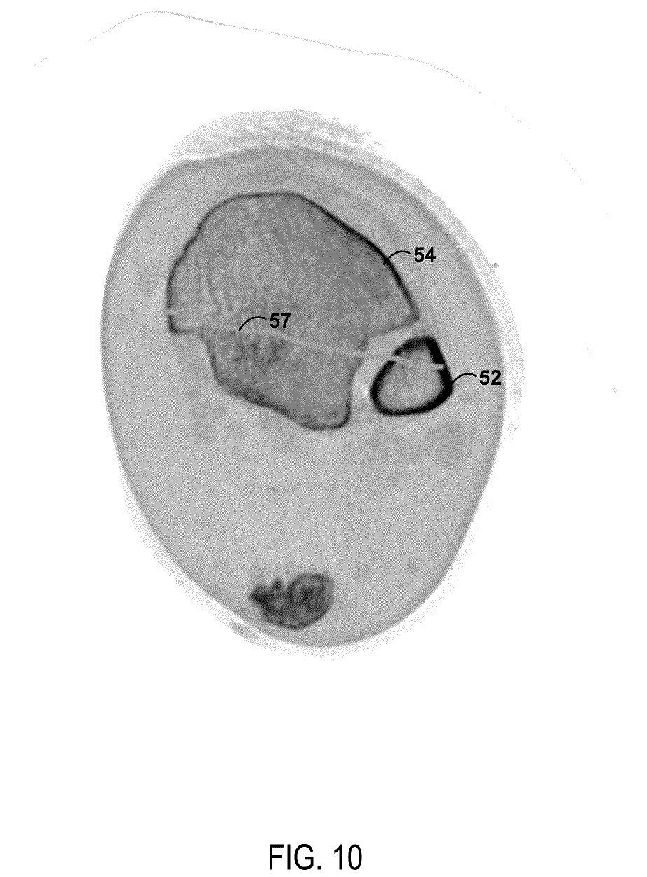

FIG. 10 shows the fibula 52 and the tibia 54. The line 57 does not coincide with the anatomical axis 21. Thus, if the guide tube 20 is aligned with the line 57, the guide pin 23 inserted through the guide tube 20 will not be properly aligned with the fibula 52 and the tibia 54.

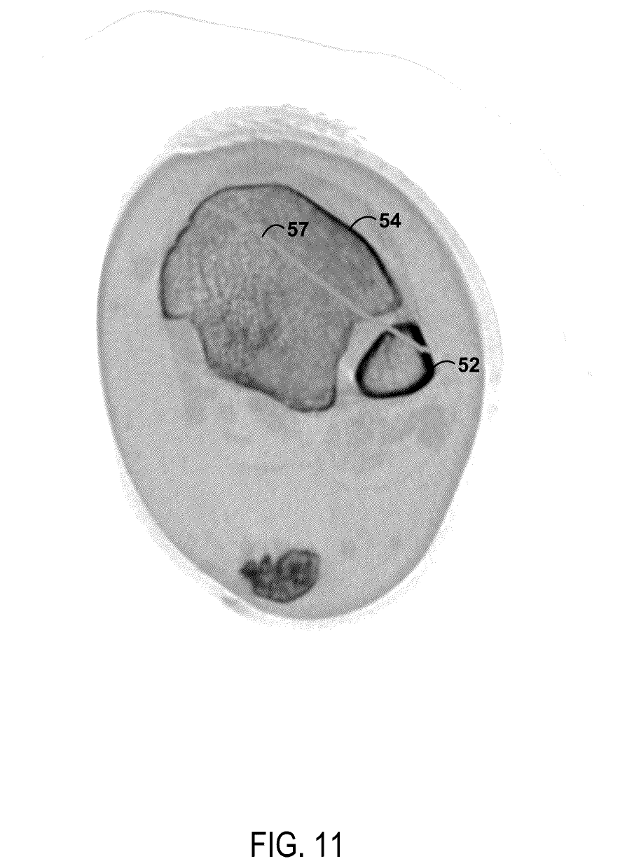

FIG. 11 shows the fibula 52 and the tibia 54. The line 57 does not coincide with the anatomical axis 21. Thus, if the guide tube 20 is aligned with the line 57, the guide pin 23 inserted through the guide tube 20 will not be properly aligned with the fibula 52 and the tibia 54.

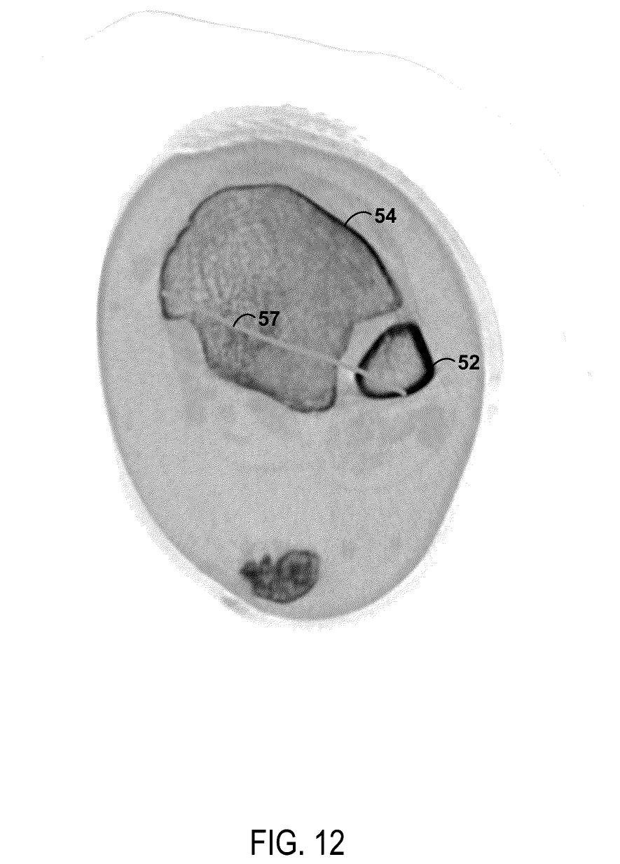

FIG. 12 shows the fibula 52 and the tibia 54. The line 57 does not coincide with the anatomical axis 21. Thus, if the guide tube 20 is aligned with the line 57, the guide pin 23 inserted through the guide tube 20 will not be properly aligned with the fibula 52 and the tibia 54.

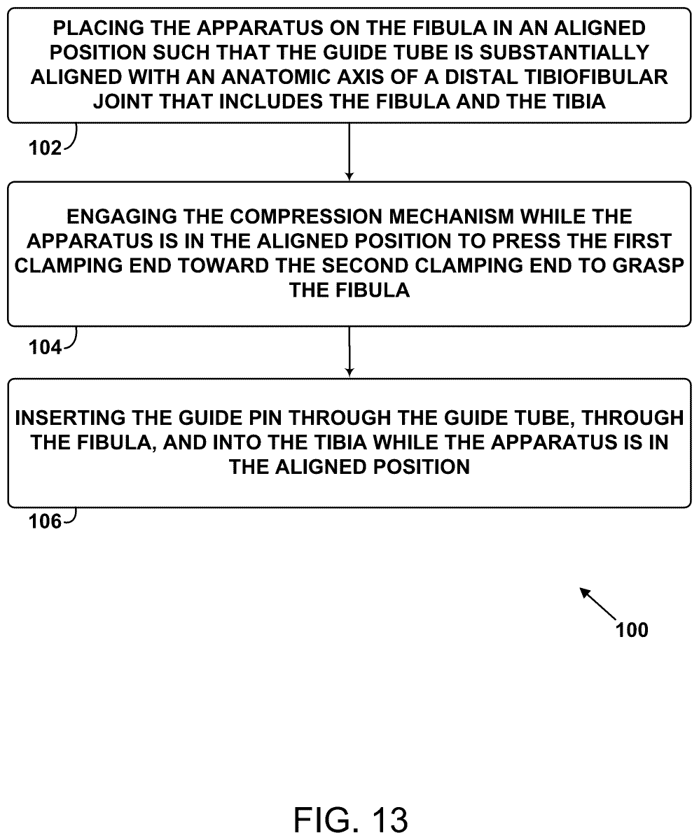

FIG. 13 is a block diagram of a method 100, which in some examples involves the apparatus 10. As shown in FIG. 13, the method 100 includes one or more operations, functions, or actions as illustrated by blocks 102, 104, and 106. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 102, the method 100 includes placing the apparatus 10 on the fibula 52 in the aligned position such that the guide tube 20 is substantially aligned with the anatomic axis 21 of the distal tibiofibular joint that includes the fibula 52 and the tibia 54. Functionality related to block 102 is described above with reference to FIGS. 2-12.

At block 104, the method 100 includes engaging the compression mechanism 18 while the apparatus 10 is in the aligned position to press the clamping end 16A toward the clamping end 16B to grasp the fibula 52. Functionality related to block 104 is described above with reference to FIGS. 2-12.

At block 106, the method 100 includes inserting the guide pin 23 through the guide tube 20, through the fibula 52, and into the tibia 54 while the apparatus 10 is in the aligned position. Functionality related to block 106 is described above with reference to FIGS. 2-12.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings, the disclosure, and the appended claims. The mere fact that certain measures or features are recited in mutually different dependent claims does not indicate that a combination of these measures or features cannot be used. Any reference signs in the claims should not be construed as limiting the scope.

What is claimed is:

1. An apparatus comprising:
a base;
a first arm extending from the base and comprising a first clamping end configured to conform to a fibula;
a second arm extending from the base opposite the first arm, the second arm comprising a second clamping end configured to conform to the fibula;
a compression mechanism configured to press the first clamping end toward the second clamping end to grasp the fibula between the first clamping end and the second clamping end; and
a guide tube between the first arm and the second arm, wherein the guide tube is formed of a radiodense material and is configured to receive and direct a guide pin for insertion through the fibula and into a tibia.

2. The apparatus of claim 1, wherein the first arm and the second arm are joined together at the base.

3. The apparatus of claim 1, wherein the first clamping end and the second clamping end each have a serrated surface configured to conform to the fibula.

4. The apparatus of claim 1, wherein the compression mechanism comprises a radiolucent material.

5. The apparatus of claim 1, wherein the compression mechanism is configured for manual adjustment of a level of compression applied by the first clamping end and the second clamping end to the fibula.

6. The apparatus of claim 1, wherein the compression mechanism comprises a spring configured to press the first clamping end toward the second clamping end.

7. The apparatus of claim 1, wherein the compression mechanism comprises a screw, wherein turning the screw adjusts a level of compression applied to press the first clamping end toward the second clamping end.

8. The apparatus of claim 1, wherein the compression mechanism comprises an adjustment dial, wherein turning the adjustment dial adjusts a level of compression applied to press the first clamping end toward the second clamping end.

9. The apparatus of claim 1, wherein the guide tube is formed of stainless steel, aluminum, and/or another metal.

10. The apparatus of claim 1, wherein the base, the first arm, and/or the second arm are radiolucent.

11. A method of using the apparatus of claim 1, the method comprising:
placing the apparatus on the fibula in an aligned position such that the guide tube is substantially aligned with an anatomic axis of a distal tibiofibular joint that includes the fibula and the tibia;
engaging the compression mechanism while the apparatus is in the aligned position to press the first clamping end toward the second clamping end to grasp the fibula; and
inserting the guide pin through the guide tube, through the fibula, and into the tibia while the apparatus is in the aligned position.

12. The method of claim 11, further comprising repairing a fracture of the fibula prior to placing the apparatus on the fibula.

13. The method of claim 11, further comprising reducing a syndesmosis prior to placing the apparatus on the fibula.

14. The method of claim 11, further comprising using x-ray fluoroscopy to determine that the apparatus is in the aligned position.

15. The method of claim 11, wherein inserting the guide pin comprises drilling the guide pin through the guide tube, through the fibula, and into the tibia such that the guide pin extends distally from the fibula.

16. The method of claim 15, further comprising:
drilling out the guide pin through the fibula and into the tibia to create a first cavity in the fibula and a second cavity in the tibia; and
binding the fibula to the tibia using a fixation device that is inserted into the first cavity and the second cavity.

17. The method of claim 16, wherein the fixation device comprises a suture.

18. The method of claim 16, wherein the fixation device comprises a screw.

19. The method of claim 16, further comprising, prior to drilling out the guide pin, placing a surgical plate on the fibula such that the guide pin extends through a hole in the surgical plate.

20. The method of claim 19, wherein placing the surgical plate on the fibula comprises placing the surgical plate on the fibula such the guide pin extends through a guide sleeve within the hole in the surgical plate.
